(19) United States
(12) Patent Application Publication
(10) Pub. No.: US 2007/0179450 A1
(43) Pub. Date: Aug. 2, 2007

(54) IRRIGATION DEVICE

(75) Inventors: John MacRae, Carlisle, ON (CA); Steven Duffield, Burlington, ON (CA)

Correspondence Address: VALENTINE A COTTRILL, 50 QUEEN STREET NORTH, STE. 1020, P.O. BOX 2248, KITCHENER, ON, N2H6M2, CA

(73) Assignee: EARIGATE INC., Burlington (CA)

(21) Appl. No.: 11/669,309

(22) Filed: Jan. 31, 2007

Related U.S. Application Data
(60) Provisional application No. 60/763,392, filed on Jan. 31, 2006.

(51) Int. Cl. A61M 5/00

(52) U.S. Cl. 604/187

(57) ABSTRACT

An irrigation device for injecting a fluid into a channel formed in an object relative to a surface of the object for washing walls defining the channel. The walls intersect to form at least one terminal portion disposed distal to the surface. The irrigation device includes an elongate body extending between an inward end and an outward end. The body includes a nozzle positioned at the inward end, and an input duct extending between the outward end and the inward end and in fluid communication with the nozzle for directing the fluid to the nozzle. The nozzle includes a plurality of output ducts in fluid communication with the input duct. The output ducts are positioned to direct the fluid out of the body substantially toward the walls and substantially toward the surface upon positioning the nozzle proximal to the terminal portion of the channel.

This application claims the benefit of U.S. Provisional Application Ser. No. 60/763,392, filed Jan. 31, 2006.

### FIELD OF THE INVENTION

This invention is related to an irrigation device for dispensing a fluid to wash a wound.

### BACKGROUND OF THE INVENTION

Irrigation devices for irrigating open wounds are known. In general, an elongate open wound forms a channel in an object relative to a surface, i.e., the patient's skin. Typically, the channel is defined by walls. The walls intersect at one or more terminal portions, determining the maximum extent of the channel relative to the surface. Wounds tend to heal from the inside out (i.e., healing tends to occur at the terminal portion), so that over time, the channel gradually becomes shallower as the wound heals. Due to such healing, therefore, the position of the terminal portion relative to the surface tends to change over time—i.e., the terminal portion gradually is moved toward the surface. In general, open wounds are irrigated (e.g., to remove material accumulated therein) as gently as possible, to minimize patient discomfort. Removal of material accumulated in the channel is desirable to minimize the possibility of infection and to promote healing.

Usually, irrigation of the wound is by means of a washing fluid directed from the outside in, i.e., generally from the surface toward the terminal portion. However, this is generally unsatisfactory, because the terminal portion and the walls of the channel often are not cleaned properly as a result. For instance, because the fluid is directed generally at the terminal portion from the vicinity of the surface (or from a point between the terminal portion and the surface), the washing fluid may not satisfactorily remove materials from the terminal portion, or the parts of the walls adjacent to the terminal portion, which are sought to be removed to permit healing to proceed.

Known irrigation devices usually include an elongate barrel portion with an outlet at a distal end thereof. The barrel portion typically is hollow and substantially cylindrical, coaxial with an axis. Also, in known devices, the outlet is usually adapted to direct fluid outwardly from the distal end, e.g., in a stream substantially aligned with the axis of the barrel. However, this structure typically results in the direction of a stream of fluid in primarily one direction into the channel (i.e., toward the terminal portion).

Yet another problem in the prior art is a tendency on the part of the user to push the irrigation device too far into the wound, thereby causing pain or discomfort to the patient. Accordingly, in order to position the irrigation device properly in the channel, a mechanism for taking into account the healing of the wound over time would be advantageous. However, prior art irrigation devices do not provide for this. The result is that the wound may be somewhat disturbed (i.e., resulting in discomfort to the patient) if the irrigation device is pushed too far into the wound in error. Alternatively, if the irrigation device is not pushed far enough into the wound, the irrigation thereby provided is not as effective as it otherwise could have been.

There is therefore a need for an irrigation device which overcomes or mitigates one or more of the deficiencies of the prior art.

### SUMMARY OF THE INVENTION

In its broad aspect, the invention provides an irrigation device for injecting a fluid into a channel formed in an object relative to a surface of the object for washing walls defining the channel. The walls intersect to form one or more terminal portions disposed distal to the surface. The irrigation device includes an elongate body extending between an inward end and an outward end. Also, the body includes a nozzle positioned at the inward end and an input duct extending between the outward end and the inward end and in fluid communication with the nozzle for directing the fluid to the nozzle. In addition, the nozzle includes a number of output ducts in fluid communication with the input duct. The output ducts are positioned to direct the fluid out of the body substantially toward the walls when the nozzle is positioned proximal to the terminal portion of the channel.

In another aspect, the body has an outer surface and the output ducts extend between the input duct and the outer surface. Each output duct terminates in a port at the outer surface. Also, each output duct includes an exit portion terminating in each port respectively. In addition, each exit portion is positioned for directing the fluid out of the port substantially toward the surface and toward the walls, to wash the walls of the channel when the nozzle is positioned in the channel.

In yet another aspect, the input duct is coaxial with a central axis substantially defining the body. Each output duct includes an exit portion which is at least partially defined by an exit portion axis respectively. Also, each exit portion axis forms an acute angle opening toward the outward end with the central axis so that each exit portion directs fluid out of the port substantially away from said at least one terminal portion and towards the walls, to wash the walls of the channel, when the nozzle is positioned proximal to the terminal portion.

In another of its aspects, the invention provides a method of injecting a fluid into the channel. The method includes the steps of, first, providing the irrigation device. Next, the nozzle is inserted into the channel to locate the nozzle a predetermined distance into the channel relative to the surface. (Because the terminal portion is spaced apart from the surface by a known (or estimated) distance, positioning the nozzle relative to the surface is, in effect, also positioning the nozzle relative to the terminal portion.) Then the fluid is provided to the input duct under pressure, and subsequently the fluid is directed into the input duct and toward the outward end. Finally, the fluid exits the output ducts and is directed thereby away from the terminal portion, to wash the walls of the channel.

In yet another of its aspects, the invention provides an irrigation device for providing a flow of a fluid in a channel formed in an object relative to a surface of the object for washing walls defining the channel. The walls intersect to form one or more terminal portions disposed distal to the surface. The irrigation device includes an elongate body extending between an inward end and an outward end, with a nozzle positioned at the inward end and an input duct extending between the outward end and the inward end and in fluid communication with the nozzle, for directing the fluid to the nozzle. The nozzle includes a number of output ducts in fluid communication with the input duct. The output ducts are positioned to direct the fluid out of the body substantially toward the surface upon the nozzle being positioned in the channel at a preselected distance relative to the surface.

In another aspect, the invention provides an irrigation device including a longitudinally extending body with an inward end and an outward end, the inward end being positionable in the channel and proximal to the terminal portion with the outward end extending out of the channel beyond the surface. The body includes an input duct extending from the outward end to the inward end and terminating in a nozzle at the inward end. The nozzle includes a number of output ducts, each output duct extending from the input duct to an outer surface of the body. The output ducts are substantially equally radially spaced apart from each other respectively, and the output ducts are formed to direct fluid out of the body and substantially toward the outward end of the body.

### BRIEF DESCRIPTION OF THE DRAWINGS

The invention will be better understood with reference to the drawings, in which:

FIG. 1 is an isometric view of an embodiment of an irrigation device of the invention;

FIG. 2 is a side view of the irrigation device of FIG. 1, drawn at a smaller scale;

FIG. 3 is a cross-section of the irrigation device of FIG. 2;

FIG. 4 is a cross-section of a nozzle of the irrigation device of FIG. 1 drawn at a larger scale; and

FIG. 5 is a cross-section of a channel of a wound in which the nozzle portion of the irrigation device of FIG. 1 is positioned, drawn at a smaller scale;

FIG. 6 is a cross-section of the channel of FIG. 5 and the irrigation device of FIG. 1, drawn at a larger scale;

FIG. 7 is an isometric view of the channel of FIG. 5 and the irrigation device of FIG. 1, drawn at a smaller scale; and

FIG. 8 is a side view of an alternative embodiment of an irrigation device of the invention.

### DETAILED DESCRIPTION

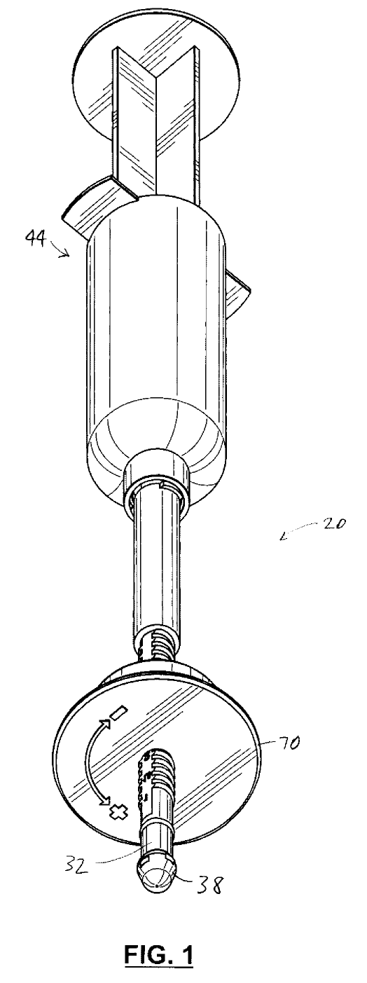

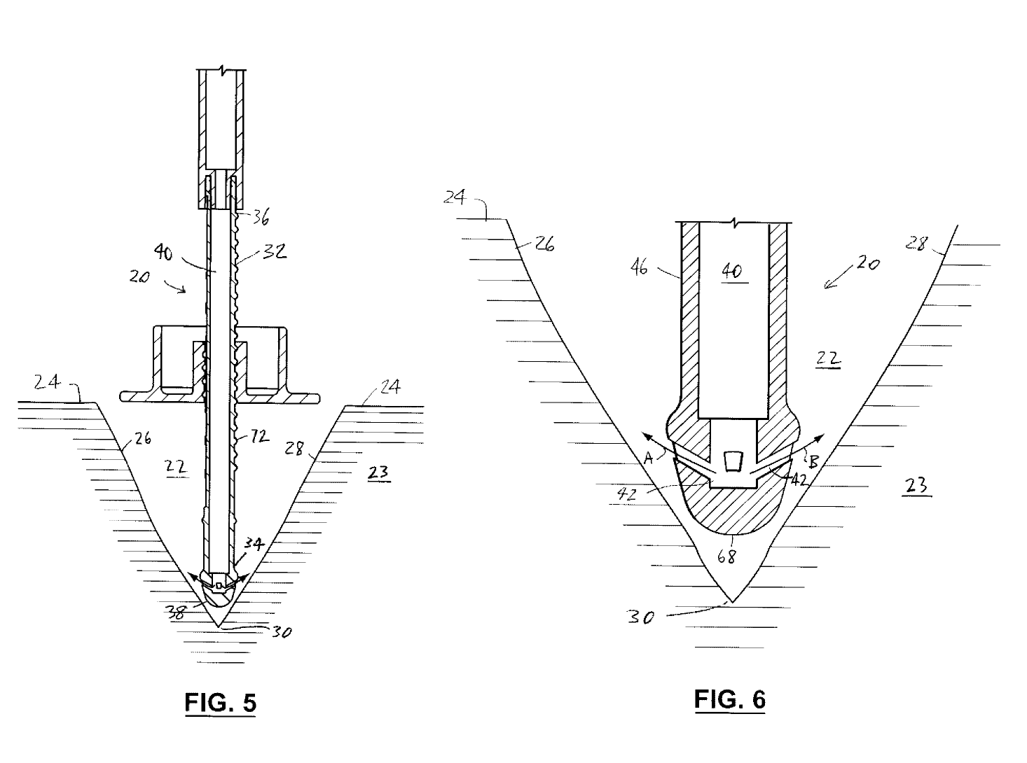

Reference is made to FIGS. 1-6 to describe an embodiment of an irrigation device in accordance with the device indicated generally by the numeral 20. The irrigation device 20 is for injecting a fluid (not shown) into a channel 22 formed in an object 23 relative to a surface 24 for washing walls 26, 28 defining the channel 22 (FIGS. 5, 6). As can be seen in FIGS. 5 and 6, the walls 26, 28 intersect to form one or more terminal portions 30 disposed distal to the surface 24. Preferably, the irrigation device 20 includes an elongate body 32 extending between an inward end 34 and an outward end 36, and the body 32 also includes a nozzle 38 positioned at the inward end 34. It is also preferred that the body 32 includes an input duct 40 extending between the outward end 36 and the inward end 34 which is in fluid communication with the nozzle 38, for directing the fluid to the nozzle 38. In one embodiment, the nozzle 38 includes a plurality of output ducts 42 in fluid communication with the input duct 40. The input ducts 42 preferably are positioned to direct the fluid out of the body 32 substantially toward the walls 26, 28 upon positioning the nozzle 38 proximal to the terminal portion 30.

Preferably, the input duct 40 is adapted for fluid communication with a reservoir or source of the fluid from which the fluid is provided under pressure substantially greater than ambient atmospheric pressure. Those skilled in the art will appreciate that various means for supplying the fluid under pressure may be used. However, it is preferred that the irrigation device 20 additionally includes a syringe subassembly 44 which is in fluid communication with the input duct 40 for supplying the fluid to the input duct 40 under pressure, as will be described.

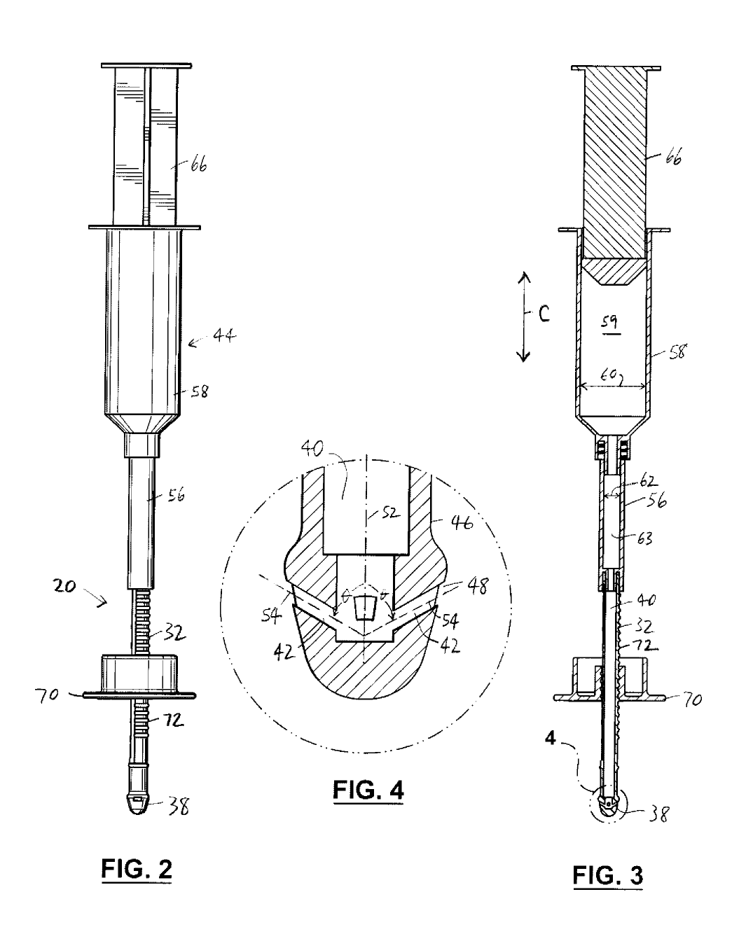

It is also preferred that the output ducts 42 are substantially radially equidistant from each other. As can be seen in FIGS. 2-4, the body 32 has an outer surface 46. Preferably, the output ducts 42 extend between the input duct 40 and the outer surface 46, and each output duct 42 terminates in a port 48 at the outer surface 46 through which the fluid is flowable. Each output duct 42 preferably is positioned for directing the fluid through the port 48 therefor (e.g., in the directions indicated by arrows “A” and “B” in FIG. 6) and substantially away from the terminal portion 30 and substantially toward the walls 26, 28, to wash the walls 26, 28.

As can be seen in FIGS. 3 and 4, the input duct 40 preferably is coaxial with a central axis 52 defining the body 32, which (in the preferred embodiment) is generally cylindrical except for the nozzle portion 38 thereof. Also, it is preferred that each output duct 42 is defined by an output duct axis 54 (FIG. 4). Each output duct axis 54 forms an acute angle θ opening toward the outward end 36 with the central axis 52 so that each output duct 42 directs fluid out of the port 48 substantially away from the terminal portion 30 and toward the walls 26, 28, to wash the walls. Preferably, the angle θ is approximately 60°.

In use, the nozzle 38 is inserted into the channel 22 until the nozzle 38 is positioned so that it is spaced apart from the surface 24 by a predetermined distance. As can be seen in FIGS. 5-7, when the nozzle 38 is in the desired position, it is preferably relatively close to the terminal portion 30. The body 32 may be positioned at any desired angle relative to the terminal portion 30. For example, the body 32 may be positioned so that it is substantially orthogonal to the terminal portion 30. The fluid is provided to the input duct 40 under pressure, and flows from the input duct 40 to the output ducts 42. The fluid is directed out of the output ducts 42 in the nozzle 38 away from the terminal portion 30 when the nozzle 38 is positioned and ready for use (i.e., relatively close to the terminal portion 30). Upon exiting through the ports 48, the fluid washes the walls 26, 28.

Preferably, the fluid is provided under pressure using the syringe assembly 44. As can be seen in FIGS. 2 and 3, the syringe assembly 44 includes a hollow syringe barrel 56 and a hollow housing 58 (to which the barrel 56 is attached) having a cavity 59 with a larger internal diameter 60 than an internal diameter 62 of a bore 63 in the syringe barrel 56. In view of the difference in the internal diameters 60, 62, the fluid positioned in the cavity 59 in the housing 58 is pressurized when a plunger 66 is pushed into the cavity 64 and forces the fluid into the bore 63. (It will be understood that the plunger 66 is movable relative to the housing 58 between inserted and extended positions (i.e., in the directions indicated by arrow “C” in FIG. 3), as is known in the art. For convenience, the plunger 66 is shown only in the extended position). Because the syringe barrel 56 is attached to the outward end 36 of the body 32 and the bore 63 of the syringe barrel 56 is directly connected with the input duct 40, the pressurized fluid flows from the syringe barrel 56 into the input duct 40 when the plunger 66 is pushed into the cavity 59. The fluid, because it is under pressure, moves relatively rapidly out of the body 32 through the output ducts 42, to spray the walls 26, 28 and thereby clean the walls.

As can be seen in FIGS. 5 and 6, when the device 20 is positioned in the channel 22, the fluid is directed out of the output ducts 42 and substantially toward the surface 24. Preferably, a tip portion 68 of the nozzle 38 is positioned proximal to the terminal portion 30. In practice, the minimum distance between the nozzle tip portion 68 which is feasible is determined by the narrowness of the channel 22 in the vicinity of the terminal portion 30. For the comfort of the patient, the device 20 preferably should be inserted so that the nozzle 38 does not engage the walls 26, 28 and/or the terminal portion 30.

It will be appreciated that any suitable fluid may be used in the device 20. Those skilled in the art would be aware of the various fluids which may be utilized in cleaning wounds and which would be suitable for use in the device 20. For example, a saline solution may be utilized, as would be known by those skilled in the art.

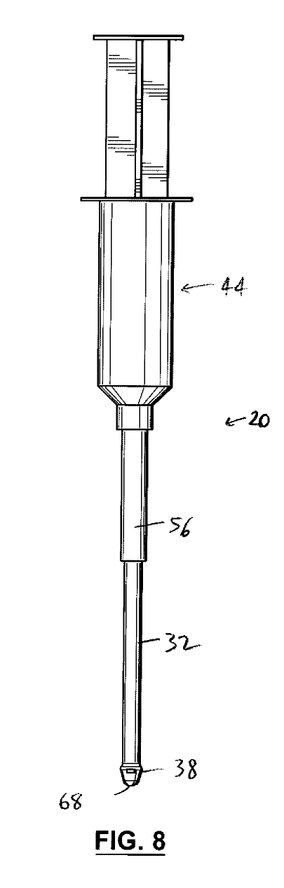

As described above, the irrigation device 20 is usable without a stop portion (FIG. 8). However, it is preferred that the irrigation device 20 additionally includes an adjustable stop portion 70 which is positionable in a range of locations relative to the nozzle 38, to position the nozzle 38 at a preselected distance from the surface 24 when the nozzle 38 is in the channel 22. Preferably, the adjustable stop portion 70 is threadably engaged with a threaded portion 72 of the body 32 and movable along the body 32 while threadably engaged therewith. The manner in which the adjustable stop portion 70 is mounted on the outer surface of the barrel is important because the threadable engagement is such that it permits the adjustable stop portion 70 to be positioned accurately relative to the nozzle 38. Also, the threadable engagement is sufficiently stiff that the adjustable stop portion 70 is not easily movable (e.g., through inadvertence) from a particular location on the body 32. (It will be understood that the irrigation device 20 shown in FIG. 8 is the same as the irrigation device shown in FIGS. 1-7, except that the irrigation device shown in FIG. 8 does not include the stop portion 70 or the threaded portion 72.)

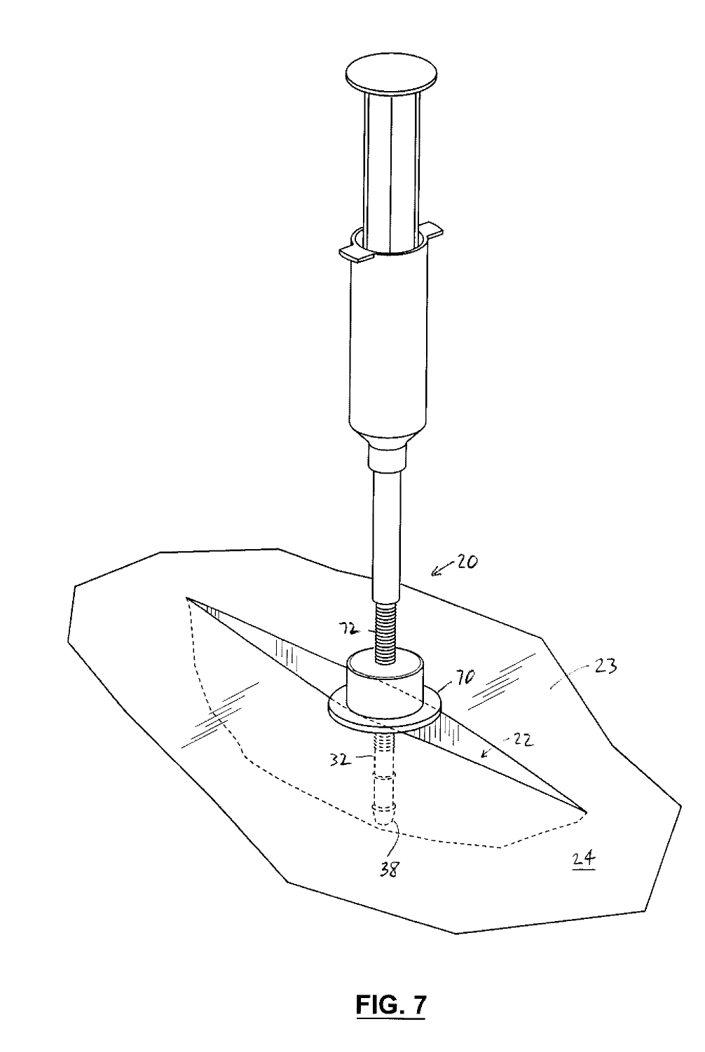

In use, the adjustable stop portion 70 is positioned relative to the nozzle tip 68 based on measurements of the channel 22. After the wound has begun to heal, the positioning of the stop portion 70 relative to the nozzle tip 68 may also be based on an estimate of the extent to which the terminal portion may have moved toward the surface due to healing of the wound. After the adjustable stop portion 70 has been moved to a desired position on the threaded portion 72, the device 20 is used. The user inserts the nozzle 38 and the body 32 into the channel 22 until the stop portion 70 engages the surface 24 at the top of the channel 22, as drawn in FIG. 7. (It will be understood that, depending on the location of the wound, the surface 22 may be positioned below or beside the terminal portion 30). Fluid under pressure is then provided to the input duct 40, and the fluid consequently exits the output ducts 42 directed substantially toward the surface, and washing the walls.

It will be appreciated by those skilled in the art that the invention can take many forms, and that such forms are within the scope of the invention as claimed. Therefore, the spirit and scope of the appended claims should not be limited to the descriptions of the preferred versions contained herein.

We claim:

1. An irrigation device for injecting a fluid into a channel formed in an object relative to a surface of the object for washing walls defining the channel, the walls intersecting to form at least one terminal portion disposed distal to the surface, the irrigation device comprising:
an elongate body extending between an inward end and an outward end;
the body comprising a nozzle positioned at the inward end;
the body comprising an input duct extending between the outward end and the inward end and in fluid communication with the nozzle for directing the fluid to the nozzle; and
the nozzle comprising a plurality of output ducts in fluid communication with the input duct, said output ducts being positioned to direct the fluid out of the body substantially toward the walls when the nozzle is positioned proximal to said at least one terminal portion of the channel.

2. An irrigation device according to claim 1 in which the input duct is adapted for fluid communication with a source of the fluid from which the fluid is provided under pressure substantially greater than ambient atmospheric pressure.

3. An irrigation device according to claim 1 additionally comprising an adjustable stop portion positionable relative to the nozzle, for positioning the nozzle in the channel at a preselected distance from the surface.

4. An irrigation device according to claim 3 in which the adjustable stop portion is threadably engaged with the body and positionable relative to the nozzle.

5. An irrigation device according to claim 1 additionally comprising a syringe subassembly in fluid communication with the input duct for providing the fluid to the input duct under pressure.

6. An irrigation device according to claim 1 in which the output ducts are substantially radially equidistant from each other.

7. An irrigation device according to claim 1 in which:
the body has an outer surface;
the output ducts extend between the input duct and the outer surface, and each said output duct terminates in a port at the outer surface;
each said output duct comprises an exit portion terminating in each said port respectively; and
each said exit portion is positioned for directing the fluid out of the port substantially toward the surface and toward the walls, to wash the walls of the channel upon the nozzle being positioned in the channel.

8. An irrigation device according to claim 1 in which:
the input duct is coaxial with a central axis substantially defining the body;
the body has an outer surface;
the output ducts extend between the input duct and the outer surface; and
each said output duct comprises an exit portion at least partially defined by an exit portion axis respectively and each said exit portion axis forms an acute angle opening towards the outward end with the central axis such that each said exit portion directs fluid out of the port substantially away from said at least one terminal portion and towards the walls, to wash the walls of the channel.

9. An irrigation device according to claim 8 in which the acute angle between each said exit portion axis and the central axis is approximately 60°.

10. A method of injecting a fluid into a channel formed in an object relative to a surface for washing walls defining the channel, the walls intersecting to form at least one terminal portion disposed distal to the surface, the method comprising:
(a) providing an irrigation device comprising:
an elongate body extending between an inward end and an outward end;
the body comprising a nozzle positioned at the inward end;
the body comprising an input duct extending between the outward end and the inward end and in fluid communication with the nozzle for directing the fluid to the nozzle;
the nozzle comprising a plurality of output ducts in fluid communication with the input duct, said output ducts being positioned to direct the fluid out of the body substantially toward the walls and substantially toward the surface when the nozzle is positioned proximal to said at least one terminal portion of the channel;
(b) inserting the nozzle into the channel to locate the nozzle a predetermined distance into the channel relative to the surface;
(c) providing the fluid to the input duct under pressure;
(d) directing the fluid into the input duct at the inward end thereof toward the outward end; and
(e) permitting the fluid to exit the output ducts, to wash the walls of the channel.

11. A method according to claim 10 in which the fluid under pressure is supplied to the input duct via a syringe subassembly.

12. A method according to claim 10 in which the irrigation device additionally comprises an adjustable stop positionable relative to the nozzle, and the method additionally comprises the following step after step (a):
(a.1) adjusting the adjustable stop to locate the nozzle at the predetermined distance relative to the surface.

13. An irrigation device for providing a flow of a fluid in a channel formed in an object relative to a surface of the object for washing walls defining the channel, the walls intersecting to form at least one terminal portion disposed distal to the surface, the irrigation device comprising:
an elongate body extending between an inward end and an outward end;
the body comprising a nozzle positioned at the inward end;
the body comprising an input duct extending between the outward end and the inward end and in fluid communication with the nozzle for directing the fluid to the nozzle; and
the nozzle comprising at least one output duct in fluid communication with the input duct, said at least one output duct being positioned to direct the fluid out of the body substantially toward the surface upon the nozzle being positioned in the channel at a preselected distance relative to the surface.

14. An irrigation device according to claim 13 in which the input duct is adapted for fluid communication with a source of the fluid from which the fluid is provided under pressure substantially greater than ambient atmospheric pressure.

15. An irrigation device according to claim 13 additionally comprising an adjustable stop portion positionable relative to the nozzle, for positioning the nozzle in the channel at a preselected distance from the surface.

16. An irrigation device according to claim 15 in which the adjustable stop portion is threadably engaged with the body and positionable relative to the nozzle.

17. An irrigation device according to claim 13 additionally comprising a syringe subassembly in fluid communication with the input duct for providing the fluid to the input duct under pressure.

18. An irrigation device according to claim 13 in which the output ducts are substantially radially equidistant from each other.

19. An irrigation device for injecting a fluid into a channel formed in an object relative to a surface of the object for washing walls defining the channel, the walls intersecting to form at least one terminal portion disposed distal to the surface, the irrigation device comprising:
a longitudinally extending body including an inward end and an outward end, the inward end being positionable in the channel and proximal to said at least one terminal portion with the outward end extending out of the channel beyond the surface;
the body comprising an input duct extending from the outward end to the inward end and terminating in a nozzle at the inward end;
the nozzle comprising a plurality of output ducts, each said output duct extending from said input duct to an outer surface of the body;
the output ducts being substantially equally radially spaced apart from each other respectively; and
said output ducts being formed to direct fluid out of the body and substantially toward the outward end of the body.

20. An ear irrigation device according to claim 19 additionally including a stop portion for positioning the nozzle in a predetermined position proximal to said at least one terminal portion, the stop portion extending transversely between the inward end and the outward end to abut the surface when the inward end is proximal to said at least one terminal portion.
